(19) United States
(12) Patent Application Publication
(10) Pub. No.: US 2025/0325726 A1
(43) Pub. Date: Oct. 23, 2025

(54) RADIOPAQUE GLASS RADIOEMBOLIZATION MICROPARTICLES AND RELATED METHODS

(71) Applicant: VARIAN MEDICAL SYSTEMS, INC., Palo Alto, CA (US)

(72) Inventors: Lingbing Lynn ZHAO, Torrance, CA (US); Theodore JACKSON, Ellicott City, MD (US)

(21) Appl. No.: 18/865,476

(22) PCT Filed: May 15, 2023
(86) PCT No.: PCT/US2023/022264

§ 371 Date: Nov. 13, 2024

Related U.S. Application Data
(60) Provisional application No. 63/342,446, filed on May 16, 2022.

(51) Int. Cl. A61L 24/00; A61K 51/12; A61L 24/02

(52) U.S. Cl. CPC A61L 24/001; A61K 51/1241; A61L 24/02; A61K 2121/00

(57) ABSTRACT

A method of performing a radioembolization treatment includes injecting 402 a plurality of radioembolization particles into a bloodstream of a patient to treat a target tissue. Each radioembolization particle of the plurality of radioembolization particles includes a radioactive core and a radiopaque layer. The method also includes obtaining 404 an image of the target tissue and the radioembolization particles to determine 406 a dose of radioactivity delivered to the target tissue by the plurality of radioembolization particles. wherein the image is one of a computerized tomography (CT) image and an x-ray image.

### CROSS REFERENCE TO RELATED APPLICATIONS

This application claims priority to and the benefit of U.S. Provisional Patent Application No. 63/342,446, filed May 16, 2022, which is hereby incorporated by reference for all purposes as if fully set forth herein.

### FIELD

The present disclosure relates to radiopaque glass radioembolization microparticles and related methods.

### BACKGROUND

This section provides background information related to the present disclosure which is not necessarily prior art.

Radioembolization can be used to treat various conditions such as cancers or other abnormal tissue growth. Radioembolization can be performed to treat abnormal tissue growth located in the liver of a patient. Radioembolization can incorporate both embolization and radiation therapies. The treatment may limit growth of the target tissue, reduce the size of target tissue, and/or destroy cells of the target tissue.

Radioembolization typically includes the introduction of particles into the blood stream of a patient that are positioned at or near the target tissue (e.g., tumor). The particles may occlude the blood vessels at the target tissue reducing or preventing blood flow to the target tissue. In addition, the particles may include a radioactive isotope that delivers a dose of radiation to the target tissue. The reduction and/or prevention of blood flow to the target tissue and the dose of radiation to the target tissue may destroy the target tissue, shrink the size of the target tissue, and/or reduce growth of the target tissue.

Existing radioembolization particles and methods of use suffer from various drawbacks. Existing radioembolization particles are difficult to position in a desired location. In addition, it can be difficult to determine a dose of radiation that is delivered to the target tissue using existing radioembolization particles. Existing radioembolization particles may therefore be positioned in sub-optimal and/or unknown locations during a treatment procedure and may deliver unknown radiation doses. Such drawbacks may lead to ineffective treatments, sub-optimal treatments, and/or to radiation doses being delivered to healthy tissues. There exists a need, therefore, for improved radioembolization particles that can be accurately and repeatedly positioned in a desired location to deliver effective radiation doses to target tissue while minimizing harmful effects to healthy tissue.

### SUMMARY

This section provides a general summary of the disclosure, and is not a comprehensive disclosure of its full scope or all of its features.

In some embodiments of the present disclosure, radioembolization particles are provided that may be used during radioembolization treatments to improve the ability of the medical professional to accurately understand the location of the radioembolization particles during the treatment process. With this information, the medical professional can take corrective and/or remedial actions to increase or improve the effectiveness of the treatment. The radioembolization particles of the present disclosure may include an additive and/or coating to make the radioembolization particles radiopaque to imaging devices used in the clinical setting where the particles are injected into the target tissue.

The radioembolization particles and related methods of use of the present disclosure are improvements over existing particles and methods. The particles and methods of use of the present disclosure can be viewed using imaging devices that can be used in the clinical setting of the treatment. Since the radioembolization particles of the present disclosure are radiopaque, the medical professionals and/or systems used in the clinical setting can determine an accurate location of the radioembolization particles and/or determine an accurate understanding of the radiation dose that will be delivered by the particles. This information can be used to improve the effectiveness of the treatment in destroying the target tissue and/or shrinking or reducing the growth of the target tissue. This information can also allow the radioembolization particles to be accurately positioned so as to limit undesirable effects on healthy tissue.

In some embodiments of the present disclosure, a radioembolization particle is provided. The radioembolization particle may include a radioactive core comprising Yttrium and Silicon and a radiopaque additive comprising at least one of Holmium, Samarium, Iodine, Iridium, Rhenium, and Indium.

In some embodiments of the present disclosure, a radio embolization particle may include a radioactive core comprising Yttrium and Silicon and a radiopaque layer applied to the radioactive core, wherein the radiopaque layer comprises a Tantalum and Bismuth coating.

In some embodiments of the present disclosure, a radioembolization treatment is provided. The radioembolization treatment may include injecting a plurality of radioembolization particles into a bloodstream of a patient to treat a target tissue. Each radioembolization particle of the plurality of radioembolization particles may include a radioactive core and a radiopaque additive or a radiopaque layer. The method may also include obtaining an image of the target tissue and the radioembolization particles to determine a dose of radioactivity delivered to the target tissue by the plurality of radioembolization particles, wherein the image includes one of a computerized tomography (CT) image and an x-ray image.

In some embodiments, a radioembolization particle is provided. The radioembolization particle may include a radioactive material that includes Yttrium and Silicon, and a radiopaque material.

In one aspect, the radiopaque material may be an additive comprising at least one of Holmium, Samarium, Iodine, Iridium, Rhenium, and Indium.

In another aspect, the radioactive material and the radiopaque material may be mixed in the radioembolization particle.

In another aspect, the radiopaque material may be a radiopaque layer and the radioactive material may be a radioactive core. The radiopaque layer can be applied to the radioactive core and the radiopaque layer may be a Tantalum and Bismuth coating.

In another aspect, the radiopaque material may be a radiopaque layer and the radioactive material may be a radioactive core. The radiopaque layer can be applied to the radioactive core, and the radiopaque layer may be a Tantalum oxide coating.

In some embodiments, a method of performing a radioembolization treatment is provided. The method may include delivering a plurality of radioembolization particles into a bloodstream of a patient to treat a target tissue, each radioembolization particle of the plurality of radioembolization particles comprising a radioactive core and a radiopaque layer. The method may also include obtaining an image of the target tissue and the radioembolization particles to determine a dose of radioactivity delivered to the target tissue by the plurality of radioembolization particles, wherein the image comprises one of a computerized tomography (CT) image and an x-ray image.

In some embodiments, a method of making a radioembolization particle is provided. The method may include combining a radioactive material comprising Yttrium and Silicon with a radiopaque material.

In one aspect, the radiopaque material may be an additive comprising at least one of Holmium, Samarium, Iodine, Iridium, Rhenium, and Indium.

In another aspect, the additive may be mixed with glass microparticle ingredients to form radiopaque glass microparticles.

In another aspect, the radiopaque material may be a radiopaque layer applied to a radioactive core, and the radiopaque layer comprises a Tantalum and Bismuth coating or a Tantalum oxide coating.

In another aspect, the radioactive material may be Yttrium and Silicon based glass microparticles.

In another aspect, the glass microparticles may be coated with the radiopaque layer by chemical vapor deposition or spray coating.

In another aspect, the method may include depositing a plurality of glass microparticles comprising Yttrium and Silicon into a reactor to obtain the radioactive material in the form of a plurality of radioactive glass microparticles. The step of combing the radioactive material with the radiopaque material may include applying a radiopaque layer to the plurality of radioactive glass microparticles.

In another aspect, the radiopaque layer may be a Tantalum and Bismuth coating

In another aspect, the radiopaque layer may be a Tantalum oxide coating.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagram illustrating an example radioembolization system in accordance with some embodiments of the present disclosure.

FIG. 2 is a flow chart illustrating an example method of producing a radiopaque glass microsphere of the present disclosure.

FIG. 3 is a flow chart illustrating an example method of producing a radiopaque glass microsphere of the present disclosure.

FIG. 4 is a flow chart illustrating an example treatment method in accordance with some embodiments of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In various embodiments of the present disclosure, radiopaque glass radioembolization particles are provided. The radiopaque glass radioembolization particles can be used for radioembolization treatments to treat abnormal tissues in a patient. In an example radioembolization treatment, the radioembolization particles of the present disclosure may be injected into the blood stream of a patient and directed to a target tissue (e.g., a tumor). The radioembolization particles can stop and/or reduce the blood supply to the target tissue and also deliver a dose of radiation to the target tissue. The radioembolization treatment can destroy the target tissue, reduce the size of the target tissue, and/or limit growth of the target tissue.

The radioembolization particles of the present disclosure are improvements over existing radioembolization particles because the radioembolization particles of the present disclosure are radiopaque. The term radiopaque is used in the present disclosure to describe a property of the particles that makes the particles opaque to various forms of radiation such as x-rays. With this property, the radioembolization particles of the present disclosure are visible in 2D and 3D x-ray images and in beam computed tomography (CT) images.

Existing radioembolization particles are not radiopaque. Existing radioembolization particles are not visible in an x-ray-based image. X-ray imaging devices, however, are often used in a clinical setting (e.g., an operating room) where a radioembolization treatment is performed. The images of the target tissue that are obtained during treatment may show a location of tracer particle but such a tracer particle is not the particle delivering the radiation to the target tissue. Existing treatment methods and existing radioembolization particles do not provide accurate, representative information for a location of the radioembolization particles.

Existing treatment methods may include post-treatment imaging in which a location of the radioembolization particles can be determining using single photon emission computed tomography (SPECT) imaging devices and/or positron emission tomography (PET) imaging devices or other post-treatment devices. Such post-treatment devices require the patient to be moved from the clinical setting (e.g., operating room) to another location to perform such image capture. This requirement does not allow a medical professional to determine a location of the radioembolization particles during treatment so that corrective or remedial actions can be taken in real-time.

Thus, the radiopaque radioembolization particles of the present disclosure are improvements over existing particles and treatment methods by allowing imaging to be performed in the clinical setting without the need to move the patient from the operating room. X-ray devices and/or beam CT scan devices can be used in the clinical setting to provide information to the medical professional regarding a location of the radioembolization particles. Accurate and reliable dose maps can be created and determined in real-time so that the treatment can be adjusted while the patient is in the clinical setting. These improvements can result in improved effectiveness of the treatment and reduced likelihood that healthy tissues are unnecessarily harmed during treatment.

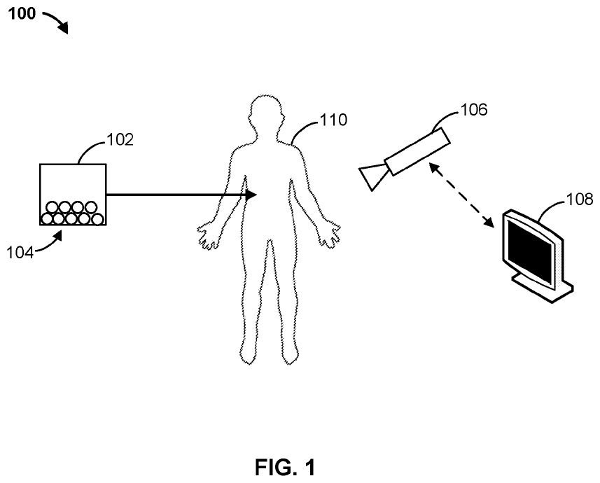

Referring now to FIG. 1, an example radioembolization system is shown. The radioembolization system may include a source of the radioembolization particles 102, an imaging device 106, and a radioembolization computing device 108. The source 102 of radioembolization particles 104 may be any suitable receptacle such as a bag, syringe, or other container that can hold the radioembolization particles 104 for delivery to a patient 110. The radioembolization particles 104 may be delivered into the bloodstream of the patient 110 using a catheter or other suitable device. The source 102 may be a syringe that can be injected with a saline or other delivery fluid.

The radioembolization particles 104 may be delivered into a predetermined vascular network of the target tissue. For example, if the radioembolization treatment is for treatment of a tumor in the liver, the liver may be imaged prior to the radioembolization treatment to determine the vasculature that delivers blood to the tumor. During the radioembolization treatment, the catheter may be positioned to deliver the radioembolization particles to this predetermined vasculature.

The radioembolization particles 104 of the present disclosure, and as will be further described below, are both radiopaque and radioactive when delivered to the target tissue of the patient 110. The imaging device 106 can be used to obtain an image of the radioembolization particles 104 in the patient 110. The location and distribution of the radioembolization particles 104 can be seen in the captured image. The imaging device 106 is a portable, x-ray-based device that can be used in the operating room or other clinical setting in which the radioembolization treatment is being performed. The imaging device 106 may be, for example, a portable x-ray device or a beam CT imaging device. Such devices may be used, traditionally, to view a location of a catheter, needle or other medical device relative to the target tissue. The radiopaque radioembolization particles of the present disclosure are also visible in the images captured by the imaging device 106.

The images obtained by the imaging device 106 may be provided to the radioembolization computing device 108. The images can be displayed or analyzed by suitable dose mapping engines or other software to determine a dose map that describes the radiation dose delivered to the target based on the location and distribution of the visible radiopaque radioembolization particles.

If the medical professional and/or the radioembolization computing device 108 determines that the radiation dose is insufficient and/or if the location and distribution of the radioembolization particles is unsatisfactory for the desired treatment, the distribution and/or location of the radioembolization can be changed and/or supplemented. Additional quantities of radioembolization particles can be delivered to the target tissue, for example. Such changes can be made to deliver satisfactory radiation doses to the target tissue and/or to prevent undesired damage to healthy tissue.

In some embodiments of the present disclosure, the radioembolization particles are glass radioembolization particles. Such particles can be made in various sizes. In some examples, the glass radioembolization particles may be generally spherical in shape and may have a diameter of about 20 to about 30 micrometers in diameter. Other suitable sizes can also be used.

The glass radioembolization particles may be made of various suitable materials. In some examples, the glass radioembolization particles are made of Yttrium and Silicon composition. The glass radioembolization particles are biocompatible to be delivered into a target tissue of a patient.

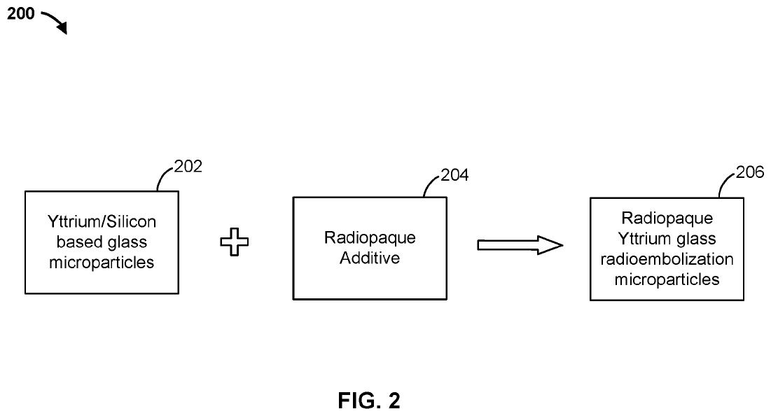

Referring now to FIG. 2, a first example radiopaque radioembolization glass microparticle process is shown. The initial Yttrium and Silicon based glass microparticle 202 can be combined with a radiopaque additive 204. The radiopaque additive 204 can be a suitable material that blocks x-ray radiation so that the radioembolization particle is visible in an x-ray based image. In various examples, the radiopaque additive 204 may include at least one of Holmium, Samarium, Iodine, Iridium, Rhenium, or Indium. The addition of the radiopaque additive results in a radiopaque Yttrium glass radioembolization microparticles 206.

Various processes for adding the radiopaque additive 204 to the glass microparticles 202 can be employed. In some embodiments, the radiopaque additive 204 is mixed with glass microparticle ingredients and radiopaque glass microparticles 206 are formed. For example. Holmium, Samarium, Iodine, Iridium, Rhenium, Indium or their oxides can be mixed and melted with the glass ingredients in a suitable oven. The mixture can then be crushed into small pieces. This composition can then be passed through spheridization equipment to form the composition into microparticles or microspheres. In such example, the radiopaque ingredients are inherently contained within the glass microparticles to result in the radiopaque glass radioembolization mircroparticles 206.

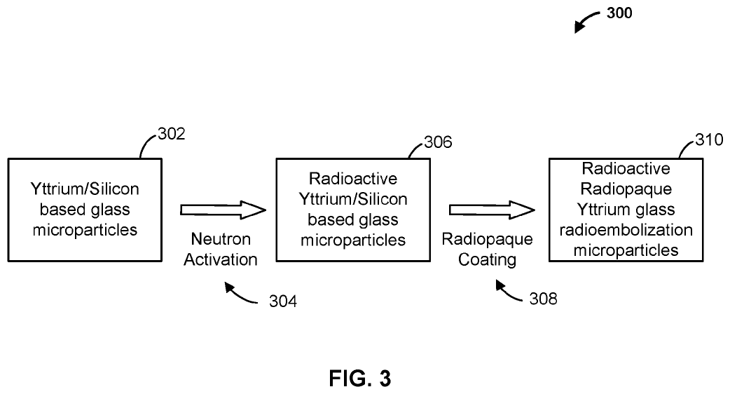

Referring now to FIG. 3, another example process 300 for producing a radiopaque glass microparticle is shown. The process 300 describes a process for coating a radioactive radioembolization particle. The process 300 may begin with a Yttrium and Silicon based glass microparticle 302 as previously described. The glass microparticles 302 can then undergo neutron activation in which the glass microparticles 302 may be deposited in a reactor and irradiated to produce Y-90 from the Y-89 contained in the glass microparticles 302. Thus, after the neutron activation 304, the microparticles 302 have been converted to radioactive Yttrium and Silicon based glass microparticles 306.

The radioactive Yttrium and Silicon based glass microparticles 306 can then be coated with a radiopaque coating 308. The radiopaque coating 308 can be various suitable coatings that are biocompatible and have a radiopaque property that can be added to the radioactive glass microparticles 306. The radiopaque coating 308 can be a layer of Tantalum oxide, for example. In another example, the radiopaque coating 308 may be a layer of Tantalum and Bismuth.

The radiopaque coating 308 can be applied to the outer surface of the radioactive glass microparticles 306. In one example, the radiopaque coating 308 can be applied to the radioactive glass microparticles 306 using a chemical vapor deposition process. In another example, the radiopaque coating 308 can be applied by a spray coating process. In such a process, the coating material (e.g., in powder form) can be melted and then sprayed using a suitable nozzle to the external surface of the radioactive glass microparticles 306. In other examples, other processes can be used to apply the radiopaque coating 308 to the radioactive glass microparticles 306.

The process 300 results in radioactive radiopaque Yttrium glass radioembolization microparticles 310. The microparticles 310 can then be delivered to clinical site for use in a radioembolization treatment. The radiopaque radioactive microparticles 310 can not only deliver the desired clinical effects of blocking blood flow and delivering radiation to a target tissue but can also be visualize during treatment in real-time using imaging devices typically available in the clinical setting.

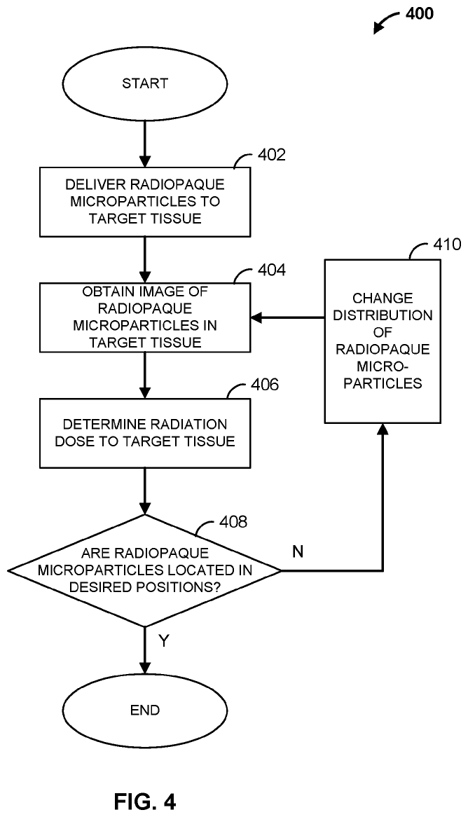

Referring now to FIG. 4, a method 400 of performing a radioembolization treatment is shown. The method 400 may utilize the radiopaque radioembolization particles previously described. The method 400 may begin at step 402 at which the radiopaque radioembolization particles are delivered to a target tissue. The particles may be delivered using the radioembolization system 100 previously described. The particles may be delivered into the bloodstream using a catheter or other suitable deliver device.

At step 404, an image is obtained of the radiopaque radioembolization particles in the target tissue. The imaging device 106 of the radioembolization system 100 can be used to obtain the image. Since the particles are radiopaque the image can show the radioactive microparticles in real-time during the delivery of the microparticles and/or during one or more intervals during the treatment process. The image can be used to determine a location, distribution and concentration of the microparticles in the target tissue.

At step 406, a radiation dose can be determined based on the image obtained at step 404. The radiation dose can be accurately determined in real-time or at a suitable point in time after delivery of the microparticles to the target tissue. The radiation dose can be determined using the radioembolization computing device 108, in some examples.

At step 408, it can be determined whether the radiopaque radioembolization microparticles are located in desired positions. The desired positions in the target tissue may be determined prior to the treatment using a diagnostic or other procedure that may analyze the vasculature of the target tissue. The desired positions may correspond to the locations of blood supply to the target tissue. The desired positions may also correspond to multiple locations of blood supply so that the radiation is delivered to target tissue. The step 408 may be performed, in some examples, by the radioembolization computing device 108 using suitable mapping and other tools. If the radiopaque radioembolization particles are located in desired positions, the method 400 may end.

If the radiopaque radioembolization particles are not located in desired positions, the method 400 may proceed to step 410. At step 410, the medical professional can take action to improve the likelihood of an effective treatment. The medical professional may, for example, change a distribution of the radiopaque radioembolization microparticles. The change may include delivery of additional radioembolization particles. An additional catheter may need to be inserted for such delivery or an additional quantity of radioembolization particles may need to be delivered at the same location.

After the change or adjustment is made at step 410, the method 400 may return to step 404 to re-perform steps 404 to 408. In this manner, the treatment can be adjusted or changed in real time while a patient is still in the clinical setting (e.g., operating room). Existing treatments require the patient to be moved to a different setting to obtain SPECT or PET images to quantify a location and/or distribution of the radioembolization particles or the radiation dose that is delivered to the target tissue. If correction is required, the patient must be moved back to the operating room for a subsequent treatment and/or a future treatment needs to be performed.

The radiopaque radioembolization particles and methods of the present disclosure are improvements over existing methods by providing real-time accurate images of the location and distribution of radioembolization particles. This improves the likelihood of an effective radioembolization treatment. The radioembolization particles of the present disclosure can also reduce the likelihood of damaging healthy tissues because the location of the radioembolization particles when being delivered to the target tissue can be imaged and visible in real-time.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A radioembolization particle comprising:
a radioactive material comprising Yttrium and Silicon; and
a radiopaque material.

2. The radioembolization particle of claim 1, wherein the radiopaque material is an additive comprising at least one of Holmium, Samarium, Iodine, Iridium, Rhenium, and Indium.

3. The radioembolization particle of claim 2, wherein the radioactive material and the radiopaque material are mixed in the radioembolization particle.

4. The radioembolization particle of claim 1, wherein the radiopaque material is a radiopaque layer and the radioactive material is a radioactive core, the radiopaque layer being applied to the radioactive core, wherein the radiopaque layer comprises a Tantalum and Bismuth coating.

5. The radioembolization particle of claim 1, wherein the radiopaque material is a radiopaque layer and the radioactive material is a radioactive core, the radiopaque layer being applied to the radioactive core, wherein the radiopaque layer comprises a Tantalum oxide coating.

6. A method of performing a radioembolization treatment comprising:
delivering a plurality of radioembolization particles into a bloodstream of a patient to treat a target tissue, each radioembolization particle of the plurality of radioembolization particles comprising a radioactive core and a radiopaque layer; and
obtaining an image of the target tissue and the radioembolization particles to determine a dose of radioactivity delivered to the target tissue by the plurality of radioembolization particles, wherein the image comprises one of a computerized tomography (CT) image and an x-ray image.

7. A method of making a radioembolization particle comprising:
combining a radioactive material comprising Yttrium and Silicon with a radiopaque material.

8. The method of claim 7, wherein the radiopaque material is an additive comprising at least one of Holmium, Samarium, Iodine, Iridium, Rhenium, and Indium.

9. The method of claim 8, wherein the additive is mixed with glass microparticle ingredients to form radiopaque glass microparticles.

10. The method of claim 7, wherein the radiopaque material is a radiopaque layer applied to a radioactive core, wherein the radiopaque layer comprises a Tantalum and Bismuth coating or a Tantalum oxide coating.

11. The method of claim 7, wherein the radioactive material comprises Yttrium and Silicon based glass microparticles.

12. The method of claim 11, wherein the glass microparticles are coated with the radiopaque layer by chemical vapor deposition or spray coating.

13. The method of claim 7, comprising:
depositing a plurality of glass microparticles comprising Yttrium and Silicon into a reactor to obtain the radioactive material in the form of a plurality of radioactive glass microparticles;
wherein the step of combing the radioactive material with the radiopaque material comprises applying a radiopaque layer to the plurality of radioactive glass microparticles.

14. The method of claim 13. wherein the radiopaque layer comprises a Tantalum and Bismuth coating

15. The method of claim 13. wherein the radiopaque layer comprises a Tantalum oxide coating.
